(19) United States
(12) Patent Application Publication
(10) Pub. No.: US 2004/0158158 A1
(43) Pub. Date: Aug. 12, 2004

(54) Method and an apparatus for localizing pulse

(76) Inventors: Martin Snejberg Jensen, Arhus (DK); Jesper Fleischer, Hojbjerg (DK)

Correspondence Address: Dykema Gossett, Suite 300 West, 1300 I Street NW, Washington, DC, 20005-3306, US

(21) Appl. No.: 10/477,352

(22) PCT Filed: May 15, 2002
(86) PCT No.: PCT/DK02/00316

(30) Foreign Application Priority Data
May 19, 2001 (DK) PA 2001 00808

(51) Int. Cl. A61B 6/00

(52) U.S. Cl. 600/476

(57) ABSTRACT

? Localization of blood vessels is based on a method and an apparatus having two or more pressure transducers, said apparatus being positioned on the skin over the blood vessel which is to be localized. The signals from the transducers are processed in accordance with the physiological point of departure by mutual weighting. The position of the blood vessel relative to the measuring transducers is found on the basis of a determination of the mass centre of the transducer signals. The transducers used are arranged in parallel in the same plane, but are physically separated by mechanical screens which prevent pressure transfer between two adjacent transducers. A measuring head in which the transducers are arranged is protected by a film of a skin-friendly material, ?ch as e.g. silicone rubber. The apparatuses for blood vessel localization visually inform the user of the position of the measuring ?aratus on the skin of the individual being measured. In an embodiment, the apparatus is used for recording the pulse in humans ?imals who or which have suffered a cardiac arrest.

The invention relates to an apparatus for localization of blood vessels and/or measurement of the pulse in humans or animals, said apparatus having a face touching the skin, said face being provided with two or more pressure-sensitive transducers, the apparatus further having a calculation unit which, on the basis of the signals from the pressure transducers, is capable of calculating the position of a blood vessel and/or measuring the pulse in a blood vessel which is positioned beneath the skin in the region in which the apparatus is disposed.

The invention moreover relates to a use.

In the present description, the term blood vessel is used for both arteries and veins, unless specifically stated otherwise.

When a medicament is to be injected via a hypodermic needle, a blood sample is to be taken, or a catheter is to be inserted into a blood vessel, it is a prerequisite, of course, that the blood vessel has been localized prior to the insertion.

One of the most frequent operations involving the need for localizing a blood vessel, concerns insertion of a catheter into arterie radialis, said operation being typically carried out near the patient's wrist.

In the previously known art, the normal procedure for localizing the artery into which the catheter is to be inserted is that the operator who is to insert the catheter, with his fingertips touches the skin at the wrist in the region beneath which the artery is presumed to be present.

Using the tactile sense, the operator now searches for the beats of the pulse which can be felt from the artery through the skin, and moves the fingertips until the strongest pulse signal is recorded, following which the catheter is inserted into the skin above the point which is presumed to be the centre of the artery.

This manual localizing process involves a relatively great percentage of error, which has the result that the catheter is not inserted correctly into the artery, which means that the process of localizing the artery and inserting the catheter has to be repeated.

Studies have shown that in the known manual technique of artery localization an average of up to three attempts in adults and 5-10 in children is carried out before the catheter is successfully inserted correctly into arterie radialis.

As insertion of catheters is associated with discomfort or pain for most patients, misinsertion because of mislocalization of the artery is very unfortunate.

Another drawback of the prior art is that it relies on the operator's s tactile sense, as the localization takes place by touch from the operator's fingertips. The localization process is therefore widely dependent on the operator.

Besides in connection with the insertion of catheters or hypodermic needles, it is often needed to localize a blood vessel for measuring the pulse of a human or an animal. In this connection, the pulse-generating blood vessel is also found manually and recorded by means of the tactile sense from one or more fingertips. The pulse count is typically obtained by manually counting the sensed beats of the pulse over a given period of time.

Pulse-counting products are available, e.g. DE 3407775 describes a hand-held product which, after localization of the pulse-generating blood vessel, can measure and show the pulse count. However, the product has the drawback that the transducer unit is very directional, which means that the product must be held at a specific angle relative to the artery on which the measurement is to be performed, in order for the pulse result to be shown correctly. The product moreover has the drawback that the pulse count shown does not allow for statistic unreliability of the pulse display caused by e.g. heart arrhythmia.

This prior art for the recording of the pulse is based on the display of a pulse count which just gives the number of the beats of the heart per minute.

In connection with attempts at resuscitation of individuals who suffer from cardiac arrest, cardiac massage is frequently used. During the cardiac massage it is a drawback of the prior art that the known pulse meters do not show how strongly the pulse is recorded. If the person giving the cardiac massage could see how strongly the pulse is recorded, e.g. at the patient's wrist, the cardiac massage could be optimized.

An apparatus as defined in the introductory part of claim 1 is known from U.S. Pat. No. 4,802,488.

It is an object of the present invention in connection with the localization of blood vessels to show them in an expedient manner to an operator.

The object of the invention is achieved by an apparatus of the type defined in the introductory portion of claim 1, which is characterized in that the apparatus has a light-generating element adapted to show the position of the blood vessel.

Expedient embodiments of the hand-held apparatus according to claim 1, described above, are defined in claims 2-6.

As mentioned, the invention also relates to the use of the apparatus. This use is defined in claim 7.

The invention will now be explained more fully with reference to the drawings, in which

FIG. 1 shows a cross-section of a body part, such as an arm, which contains a blood vessel to be localized using an apparatus having several pressure transducers,

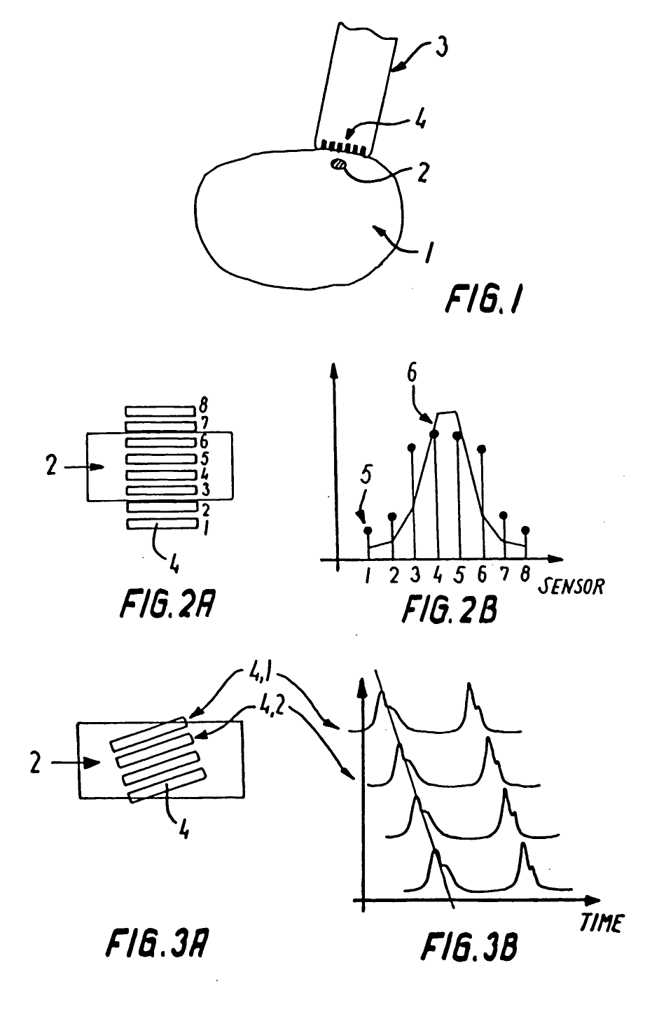

FIG. 2A shows several identical pressure transducers positioned above a pulse-generating blood vessel,

FIG. 2B shows the signals from the pressure transducers shown in FIG. 2A drawn in a system of coordinates as a function of two calculation models,

FIG. 3A shows a measuring head having several pressure transducers positioned obliquely relative to the longitudinal axis of a pulse-generating blood vessel which is positioned beneath the skin of the body part being measured.

FIG. 3B shows the signals from the transducers which are shown in FIG. 2A, depicted in a system of coordinates where the horizontal axis indicates time,

FIG. 4A shows a measuring head having several pressure transducers arranged offset relative to the longitudinal axis of the blood vessel being measured,

FIG. 4B shows the signals from the transducers drawn in 4A,

FIG. 5 shows a sectional view of a measuring head for an apparatus for localizing a blood vessel, with six identical pressure transducers positioned in a mechanical support with a mechanical shield between the individual transducers, which are all protected by a surface film,

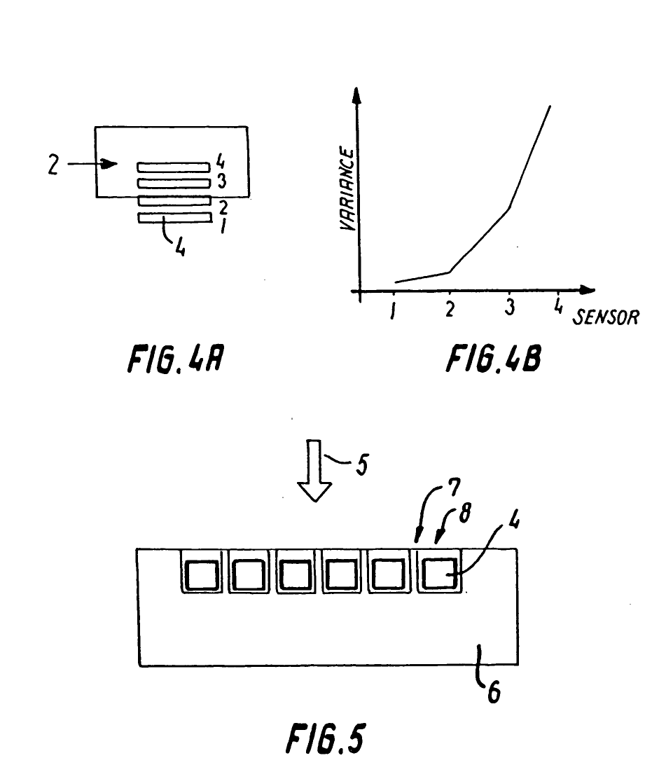

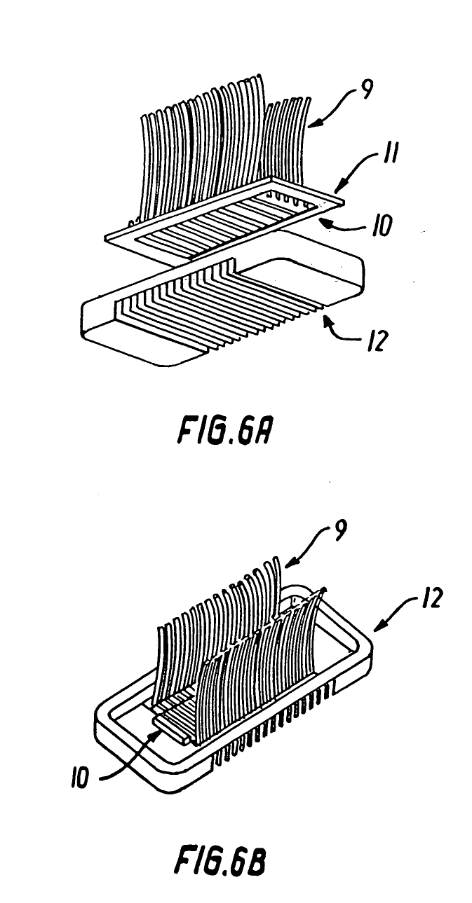

FIG. 6A shows an exploded example of a measuring head having 16 parallel transducers for localizing blood vessels,

FIG. 6B shows the same apparatus as is shown in FIG. 6A, but in an assembled state,

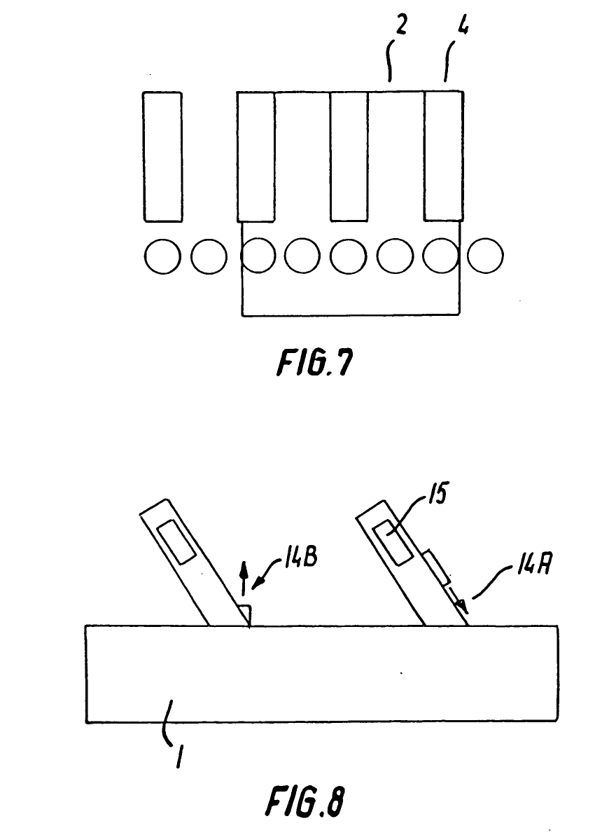

FIG. 7 is a basic sketch of a light signal indicator for localizing a blood vessel from a hand-held apparatus having several pressure transducers,

FIG. 8 is a basic sketch of two types of light indicators for positional determination of a blood vessel;

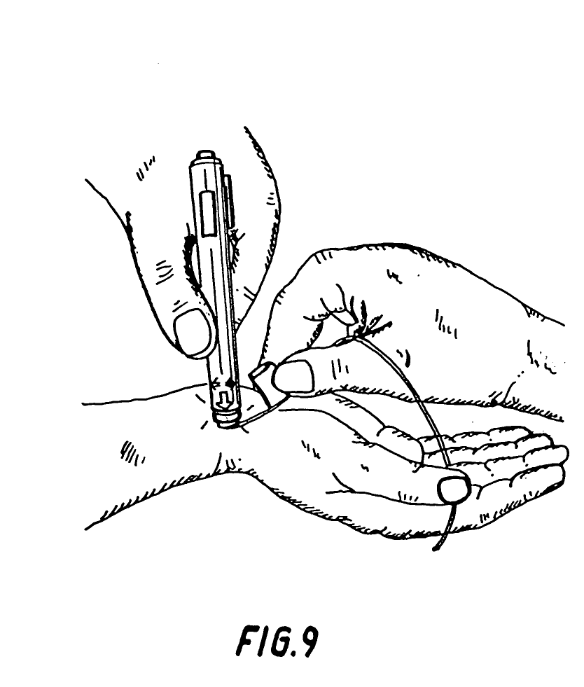

FIG. 9 is an example of the use of a hand-held apparatus for localizing blood vessels,

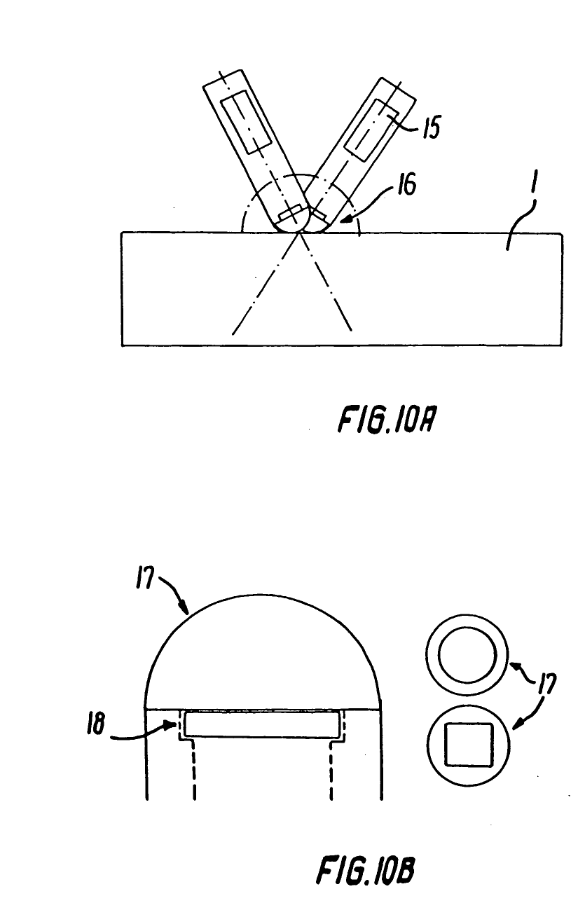

FIG. 10A shows a hand-held apparatus for pulse display having a hemispherical measuring head, said apparatus being depicted in two different angles relative to the body which includes the blood vessel being measured, while

FIG. 10B shows a section of the hemispherical measuring head from the hand-held product shown in FIG. 9A.

FIG. 1 shows a cross-section of a body part 1, e.g. an arm, which includes a blood vessel 2 which is to be localized.

The localization takes place with a hand-held apparatus 3 which is placed on the skin above the region in which the blood vessel 2 is present. The hand-held apparatus is provided with a plurality of pressure transducers 4 on the face touching the skin. In the example shown, a total of six transducers are positioned in the measuring face.

The pressure transducers can record the pressure changes which occur at each beat of the heart in the individual being measured. When the heart contracts from the end diastolic phase to the end systolic phase, the blood is pumped from the heart around in the body, which can be recorded most clearly in the arteries that carry the oxygenated blood. It is shown in FIG. 2A how eight identical pressure transducers are positioned over an artery. The transducers are arranged in parallel in the same plane and thereby have an extent that extends beyond the width of the blood vessel.

Each pressure transducer will emit a signal which is proportional to the measured pressure impact.

The pressure impact from the artery to the transducers will follow the heartbeat and be greatest for the transducers positioned most closely to the mass centre of the artery or the centre line which describes the mass centre in the longitudinal axis of the artery. The signal from the transducers will thus be greatest from the transducer or transducers which are closest to the centre line of the artery, and smallest from the transducers most remote from the centre line of the artery.

The relation between the signals from the transducers may therefore be used for deriving the position of the underlying blood vessel relative to the position of the measuring head on the skin.

The method used for analyzing the signals from the pressure transducers with a view to determining the location of a blood vessel from which the pressure impacts are recorded, is optimized from the physiological point of departure.

Initially, the actual pulse signal is searched for, based on the use of so-called cross-correlation on all the transducer signals. The result of the cross-correlation determination of the signals measured from the transducers in FIG. 2A is shown in FIG. 2B, point 5.

Subsequently, the variance of the signals from all the transducers is calculated, which for the example shown in FIG. 2A gives a result as shown in FIG. 2B, point 6.

The position of the blood vessel relative to the measuring transducers may now be derived with great accuracy by weighted comparison of the correlation and variance determinations. The centre of the blood vessel will be present at a point determined by the mass centre of the detected and processed signals.

The method is optimized by filtering-off noise signals and by comparison of the measured signals with simulated ideal data.

The weighted analysis of the signals from all the pressure transducers may also be used for the initial positioning of the measuring transducers over the blood vessel whose position is to be determined.

As shown in FIGS. 3A and 3B, time differences will occur in the signal response from the transducers, if these are rotated in their longitudinal direction relative to the longitudinal direction of the blood vessel. FIG. 3A shows a measuring transducer set which is angled relative to the longitudinal direction of the blood vessel, and the signal response from the transducers shown in FIG. 3A is depicted in FIG. 3B. It will be seen from the illustrations that the time difference between the transducer signals will diminish and approach zero, when the transducers are rotated relative to the blood vessel until the longitudinal directions of the transducers and the blood vessel extend in parallel.

FIG. 4A shows an example where the measuring transducers are positioned such that a portion of the blood vessel to be measured is not present within the region which is covered by the transducers. The signals from the set-up shown in FIG. 4A are depicted in FIG. 4B, from which it will appear that the signals from the transducers do not have a maximum among the central transducers, but that the signals have extremes, minimum and maximum respectively, from the outermost transducers.

A signal output such as the one shown in FIG. 4B clearly indicates that the measuring transducers are not positioned expediently over the blood vessel, but are positioned offset relative to it and must be moved in a direction toward the transducer that emits the greatest signal. The required movement may be shown on the hand-held apparatus for guidance of the user.

Moreover, it is part of the method according to the present invention that the time signals from the transducers are analyzed with a view to having the measuring transducers positioned expediently over the blood vessel whose position is to be detected.

The time analysis sequence is used for determining the correct angle of the measuring transducers relative to the longitudinal direction of the blood vessel whose position is to be localized.

As mentioned, the invention also relates to apparatuses, all of which are characterized by containing a measuring head having a plurality of pressure transducers for recording the pulse from a blood vessel. FIG. 5 shows a cross-sectional view of such a measuring head.

FIG. 5, the measuring head is equipped with six identical pressure transducers 4 which are mechanically mounted on a mechanical support 6 and mutually separated by a mechanical screen 7, which prevents mechanical pressure transfer between the individual transducers. The measuring head records a pressure 5 which originates from an underlying blood vessel.

To protect the measuring head and the individual transducers, the surface is coated with a thin film 8 of a skin-friendly material, such as e.g. silicon rubber.

FIGS. 6A and 6B show a measuring head having a plurality of 16 transducers 10 positioned on a support 11, which may e.g. be a printed circuit board, through which the leads 9 to the transducer elements are run. The transducers are positioned in a mechanical housing 12 constructed such that a mechanical shield is provided between each transducer.

FIG. 6B shows the same apparatus part as FIG. 6A, merely seen from another angle and with the parts assembled.

In connection with the localization of a blood vessel in accordance with the present invention, it is important that the position is shown in an expedient manner to the operator. FIG. 7 shows an example of how the position of the blood vessel may be depicted visually using light-emitting diodes or a light guide arrangement. Light-emitting elements 13 are mounted in parallel with the transducers 4, and, as shown in FIG. 7, the light-generating element present precisely over the centre line of the blood vessel 2 which has been localized, is activated.

Supplementary examples of how the position of a localized blood vessel may be shown to the operator, are illustrated in FIG. 8, where two different light paths 14A and 14B may be used.

FIG. 8 also shows how the pulse count may be shown on the hand-held apparatus.

The operator may use the light marking for marking the point on the skin where a hypodermic needle or a catheter is to be inserted.

FIG. 9 shows an example of the use of an apparatus for localizing a blood vessel relative to the present invention.

With the described method and by using an apparatus according to the present invention and as described above, it is ensured that a blood vessel may be localized simply, rapidly and distinctly, independently of the operator's tactile sense.

A product, improved over the prior art, for recording the pulse from a hand-held apparatus forms part of the present invention. The novel technique is illustrated by a basic sketch in FIG. 10A. It is characteristic of the invention that the pulse may be measured with the apparatus positioned with great degrees of freedom relative to the skin beneath which the blood vessel, from which the pulse is to be recorded, is present.

FIG. 10A shows pulse measurements with the apparatus positioned at two different angles. The reason why it is possible to measure the pulse at the different angles is that the transducer head, which is characterized by having an active measuring surface in this variant of the invention, is constructed as a hemisphere.

An enlarged section of the measuring head from FIG. 10A is shown in FIG. 10B that shows the measuring head in cross-section, from which it appears that the measuring head 17 is active in contact with the transducer unit 18 across the entire hemispherical surface.

FIG. 10B shows the hemispherical measuring head 17 seen from the end with a circular and a square, respectively, transducer element arranged behind it.

The pulse-measuring apparatus is also used in connection with cardiac massage, which is given in connection with attempts at resuscitation of humans or animals suffering from cardiac arrest. A pulse-measuring apparatus according to the present invention may be attached around e.g. the wrist of the individual who is to be resuscitated, the pulse being measured continuously from an artery disposed beneath the skin. It is characteristic of such an apparatus that it emits a continuous signal which is proportional to the pulse strength from the blood vessel being measured. This facility enables the individual performing the cardiac massage to optimize the effect of it in accordance with the signal display of the apparatus.

The transducers incorporated in the present invention may frequently advantageously be made of piezoelectric or piezoresistive crystals.

All the stated apparatuses may be provided with electronic components for signal processing and signal calculation. The apparatuses may also all be provided with expedient signal generators, including acoustic sound generators and/or displays, such as alphanumeric and/or graphic ones, for data representation.

Further, the products may be provided with electronic components for wireless data communication with external units, such as computers.

All the apparatuses comprised by the present invention may advantageously be powered from integrated batteries, which may be rechargeable.

The apparatuses may also be provided with mechanical elements for attachment to garments, such as e.g. shirt pockets.

It moreover applies to all the apparatuses that they may advantageously be made of materials which are non-toxic and tolerate cleaning as well as sterilization.

1. An apparatus (3) for localization of blood vessels (2) and/or measurement of the pulse in humans or animals, said apparatus having a face touching the skin, said face being provided with two or more pressure-sensitive transducers (4), the apparatus further having a calculation unit which, on the basis of the signals from the pressure transducers, is capable of calculating the position of a blood vessel and/or measuring the pulse in a blood vessel which is positioned beneath the skin in the region in which the apparatus is disposed, characterized in that the apparatus has a light-generating element adapted to show the position of the blood vessel.

2. An apparatus according to claim 1, characterized in that the light-generating element consists of light-emitting diodes.

3. An apparatus according to claim 2, characterized in that the light-emitting diodes are mounted in parallel with the transducers.

4. An apparatus according to claims 1-3, characterized in that the light-generating element emits light towards (14A) the skin.

5. An apparatus according to claims 1-4, characterized in that the light-generating element produces a light marking point on the skin.

6. An apparatus according to claims 1-3, characterized in that the light-generating element emits light away (14B) from the skin.

7. Use of the apparatus according to claims 1-7 in connection with cardiac massage.
